(19) United States
(12) Patent Application Publication
(10) Pub. No.: US 2002/0137679 A1
(43) Pub. Date: Sep. 26, 2002

(54) COMP/TSP-1, COMP/TSP-2 and other TSP chimeric proteins

(75) Inventor: John W. Lawler, Swampscott, MA (US)

Correspondence Address: HAMILTON, BROOK, SMITH & REYNOLDS, P.C., 530 VIRGINIA ROAD, P.O. BOX 9133, CONCORD, MA, 01742-9133, US

(73) Assignee: Beth Israel Deaconess Medical Center, Boston, MA

(21) Appl. No.: 09/919,603

(22) Filed: Jul. 30, 2001

Related U.S. Application Data
(63) Continuation of application No. PCT/US00/02482, filed on Feb. 1, 2000.
(60) Provisional application No. 60/118,053, filed on Feb. 1, 1999.

(51) Int. Cl. A61K 31/17; C07H 21/04; C12P 21/04; C12N 5/06

(52) U.S. Cl. 514/12; 514/8; 435/69.7; 530/350; 435/325; 435/320.1; 536/23.5

(57) ABSTRACT

Tumors attract blood vessels in order to grow by a process called angiogenesis. The relative quantity of stimulators and inhibitors is an important determining factor for the initiation of angiogenesis. Thrombospondins-1 and -2 are adhesive glycoproteins that have the ability to inhibit angiogenesis. This inhibiting activity has been mapped to the type 1 repeats of TSP-1 and TSP-2. The invention includes chimeric proteins that contain anti-angiogenic portions of TSP-1, TSP-2, endostatin, angiostatin, platelet factor 4, or prolactin, linked to a portion of the N-terminal region of human cartilage oligomeric matrix protein (COMP) that allows formation of pentamers. Also described herein are the nucleic acid molecules, vectors, and host cells for expressing and producing these chimeric proteins. Further embodiments of the invention include methods to treat humans or other mammals with anti-angiogenic proteins to reduce tumor size or rate of growth. Since the type 1 repeat region of TSP-1 and TSP-2 reportedly inhibits HIV infection, chimeric proteins comprising these repeats may also be used for this purpose, as well as to inhibit angiogenesis.

### RELATED APPLICATIONS

This application is a continuation of International Application No. PCT/US00/02482, which designated the United States and was filed Feb. 1, 2000, published in English, which claims the benefit of U.S. Provisional Application No. 60/118,053 filed Feb. 1, 1999. The entire teachings of the above applications are incorporated herein by reference.

### GOVERNMENT SUPPORT

The invention was supported, in whole or in part, by grants HL 28749 and HL 49081 form The Heart, Lung and Blood Institute of the National Institutes of Health. The Government has certain rights in the invention.

### BACKGROUND OF THE INVENTION

Thrombospondins are a family of calcium-binding multifunctional glycoproteins that are secreted by various cell types and are developmentally regulated components of the extracellular matrix (Bornstein, P., FASEB J., 6:3290-3299, 1992; Bornstein, P., J. Cell Biol., 130:503-506, 1995). Among their functions are modulating cell attachment, migration and proliferation.

One member of this family, cartilage oligomeric matrix protein (COMP) is a pentamer in which multimerization appears to be directed by α-helical segments situated (in the amino acid sequence) either before or after the cysteine residues that form the interchain disulfide bonds. COMP has been purified (Prochownik, E. V. et al., J. Cell Biol. 109:843-852 (1989)). Individuals affected with pseudoachondroplasia, who have considerably shortened stature as a result of premature cessation of bone growth, have been shown to have mutations in exon 17B of the COMP protein (Nature Genetics 10:325-329 (1995)).

In vitro assays have shown that platelet thrombospondin-1 is involved in thrombosis, fibrinolysis, wound healing, inflammation, tumor cell metastasis and angiogenesis. The major form of thrombospondin secreted by platelets and endothelial cells is TSP-1. Thrombospondin-1 (TSP-1) is an angiogenesis inhibitor that decreases tumor growth. Thrombospondin-2 (TSP-2) is a related glycoprotein of similar structure and properties.

The thrombospondin type 1 repeats (TSRs; also “repeat regions” herein) have been shown to inhibit angiogenesis and HIV infection. However, other portions of the proteins have been shown to have a positive effect on endothelial cell growth. Thromobospondin-1 and -2 are similar in terms of their molecular architecture. Thrombospondin-land thrombospondin-2 each have three copies of the TSR. TSP-1 and TSP-2 are trimeric molecules. Thus, each fully assembled protein contains nine TSRs.

Whereas TSP-1 and TSP-2 are antiangiogenic, these proteins contain other domains that have additional activities that diminish the antiangiogenic activity. The isolated TSRs are more potent inhibitors of angiogenesis than the native molecules.

The ingrowth of new capillary networks into developing tumors is essential for the progression of cancer. Thus, the development of pharmaceuticals that inhibit the process of angiogenesis is an important therapeutic goal. As pointed out in a review by Folkman (Folkman, J., Proc. Natl. Acad. Sci. USA 95:9064-9066, 1998), antiangiogenic therapy has little toxicity, does not require the therapeutic agent to enter tumor cells or cross the blood-brain barrier, controls tumor growth independently of growth of tumor cell heterogeneity, and does not induce drug resistance.

### SUMMARY OF THE INVENTION

The invention includes chimeric proteins comprising: (1) a chimeric protein comprising the second and third type 1 repeats of human TSP-1, and which may also comprise the procollagen homology region of TSP-1; (2) a chimeric protein comprising the multimerization domain of human COMP, the first type 2 repeat of human COMP, and the second and third type 1 repeats of human TSP-1; (3) a chimeric protein comprising the multimerization domain of human COMP, the first type 2 repeat of human COMP, and the second and third type 1 repeats of human TSP-1, but not the TGF-β activation region of human TSP-1; (4) a chimeric protein comprising the multimerization domain of human COMP, the procollagen region, and the first, second, and third type 1 repeats of human TSP-1; (5) a chimeric protein comprising the three type 1 repeats of human TSP-2, and which may also comprise the procollagen homology region of TSP-2; (6) a chimeric protein comprising the multimerization domain of human COMP, the first type 2 repeat of human COMP, and the three type 1 repeats of human TSP-2; and (7) variants of any of the above having anti-angiogenic activity. The invention further includes isolated nucleic acids encoding any of the above chimeric proteins, vectors comprising these nucleic acids, and host cells comprising any of said vectors. The chimeric proteins can be produced in host cells and used in methods for the treatment of a disease or medical condition characterized by abnormal or undesirable proliferation of blood vessels, such as that occurring in tumor growth.

### BRIEF DESCRIPTION OF THE DRAWINGS

FIG. 1 is a representation of the amino acid sequence of human TSP-1 (SEQ ID NO: 1). The type 1 repeats of TSP-1 are, as illustrated here, 1) amino acids 361-416;2) amino acids 417-473; and 3) amino acids 474-530.

FIG. 2 is a representation of the amino acid sequence of human TSP-2 (SEQ ID NO: 2). The type 1 repeats of TSP-2 are, as illustrated here, 1) amino acids 381-436; 2) amino acids 437-493; and 3) amino acids 494-550.

FIG. 3 is a representation of the amino acid sequence of human COMP (SEQ ID NO: 3). The type 2 repeats of COMP are, as illustrated here, 1) amino acids 89-128; 2) amino acids 129-181; 3) amino acids 182-226; and 4) amino acids 227-268.

FIGS. 4A and 4B together are a representation of the DNA sequence (SEQ ID NO: 4) of gene encoding a human COMP/TSP-1 chimeric protein and the amino acid sequence (SEQ ID NO: 5) of a human COMP/TSP-1 chimeric protein encoded by the DNA sequence above it.

FIG. 5A and 5B together are a representation of the DNA sequence (SEQ ID NO: 6) of a gene encoding a human COMP/TSP-2 chimeric protein and the amino acid sequence (SEQ ID NO: 7) of a human COMP/TSP-2 chimeric protein encoded by the DNA sequence above it.

FIG. 6 is a schematic representation of a few of the chimeric protein embodiments of the invention.

FIG. 7 is a graph showing tumor volume (mm³) at 7, 14 and 21 days in the experiment described in Example 3, in which mice were injected with an unaltered (control) vector, pNeo (filled diamonds) or with an expression vector encoding COMP/TSP-1 chimeric protein (filled squares).

### DETAILED DESCRIPTION OF THE INVENTION

Described herein is a protein that has the functional activity of the TSR but not other activities associated with TSP-1 or TSP-2, and is assembled into a multimeric structure. One embodiment of the invention is a chimeric protein that comprises the TSRs from TSP-1 or TSP-2 and the multimer assembly region of human cartilage oligomeric matrix protein (COMP), using a portion of the amino-terminal end. Other portions of TSP-1 or TSP-2 can be incorporated into the chimeric protein, such as the procollagen homology region of TSP-1 and/or TSP-2. The last two TSRs of TSP-1 are preferably used because the first TSR has the ability to activate transforming growth factor β (TGF-β), which stimulates tumor growth. The COMP assembly domain spontaneously forms a 5-stranded α-helical domain, allowing for the use of the COMP domain as a tool for pentamerization.

Thus, the COMP/TSP-1 construct contains the region for multimerization, the first type 2 repeat of human COMP (construct encodes amino acids 1-128) and the second and third TSRs of human TSP-1 (construct encodes amino acids 417-530). See the Table for active sequences of TSP-1 (taken from chapter 2, “The Primary Structure of the Thrombospondins” In The Thrombospondin Gene Family (J. C. Adams et al., eds.) Springer-Verlag, Heidelberg (1995)). The assembled protein is a pentamer containing 10 copies of the TSR. Thus, COMP/TSP-1 and COMP/TSP-2 are expected to be more active than TSP-1 and TSP-2. COMP/TSP-1 and COMP/TSP-2 are expected to be correctly folded and multimeric so that they better mimic the natural proteins than peptides that are based on the TSR sequence.

The first type 2 repeat of COMP includes amino acid residues 73-130, based on the genomic sequence. The amount of COMP sequence at the 3′ end can be increased or decreased to maximize activity. For example, two or more type 2 repeats of COMP can be included if moving the type 1 repeats of TSP-1 or TSP-2 farther out on the arms of the expressed protein increases its activity. Alternatively, “spacer” sequence not naturally occurring in COMP or in TSP-1 or TSP-2 can be added. The COMP/TSP-2 construct contains the same region of COMP and the three TSRs of human TSP-2 (construct encodes amino acids 381-550). When it is assembled to a pentamer this chimeric protein will contain 15 TSRs. Because these proteins are derived from portions of human proteins, they should not be immunogenic in humans.

TABLE
Active Regions of Interest Within Thrombospondin-1

| Domain | Sequence | Function |
|---|---|---|
| Procollagen | NGVQYRN (SEQ ID NO: 8) | Anti-angiogenesis |
| homology | | |
| Type 1 repeats | CSVTCG (SEQ ID NO: 9) | Cell binding |
| | WSXWSXW (SEQ ID NO: 10) | Heparin binding |
| | GGWSHW (SEQ ID NO: 11) | TGF-β and Fibronectin |
| | | binding |
| | RFK | TGF-β activation |
| | SPWDICSVTCGGGVQKRSR (SEQ ID NO: 12) | Anti-angiogenesis |
| Type 2 repeats | DVDEC(X)₆C(X)₈CENTDPGYNCLPC (SEQ ID NO: 13) | Calcium binding |

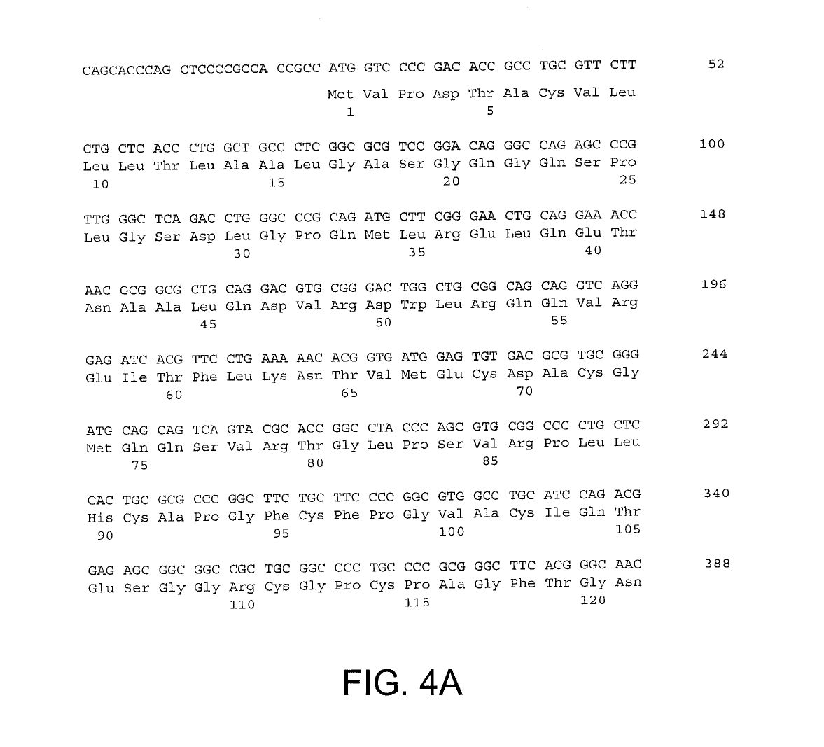

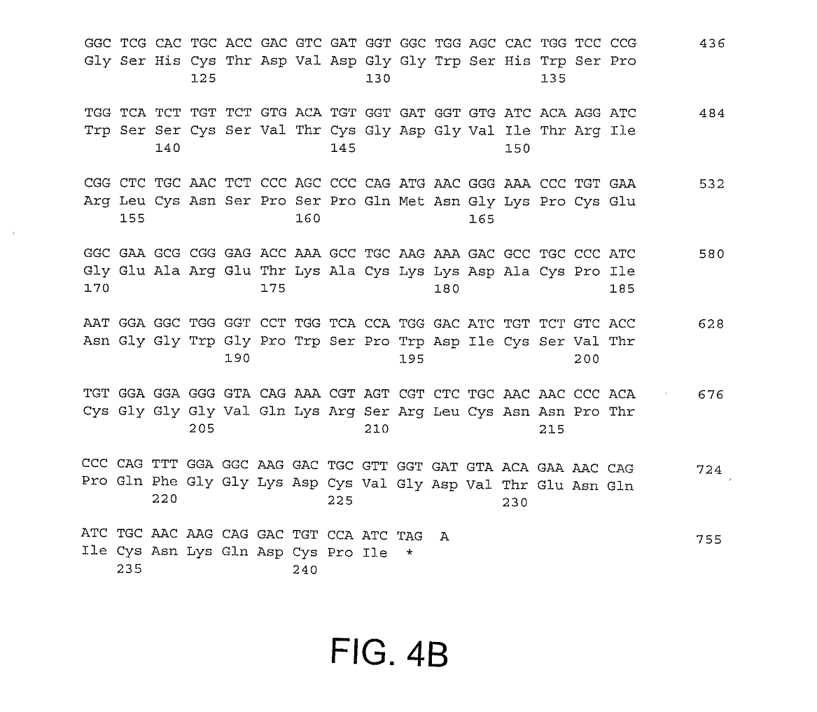

In one aspect, the invention comprises polynucleotides or nucleic acid molecules that encode chimeric proteins having portions whose amino acid sequences are derived from human TSP-1. By the genomic structure, the type 1 repeats of TSP-1 are amino acid residues 359-414 (first), amino acid residues 415-473 (second), and 474-531 (third). In one case, the chimeric protein encoded by the polynucleotides of the invention comprises the second and third type 1 repeats of human TSP-1. Such a chimeric protein may also comprise the procollagen homology region and the first type 1 repeat of TSP-1. If amino acid sequences that activate TGF-β are included in the product protein, and are found to reduce anti-angiogenic activity, the RFK sequence can be mutated (to QFK, for example) to a sequence that does not activate TGF-β, by appropriate manipulations of the nucleic acid molecule or construct encoding the chimeric proteins. In another case, the chimeric proteins encoded by the polynucleotides of the invention are variants of the immediately aforementioned chimeric protein which have activity that is similar in quality and quantity (for example, plus or minus one order of magnitude in an assay) to the anti-angiogenic activity of the protein whose amino acid sequence is represented in FIGS. 4A and 4B. In another case, the chimeric proteins encoded by polynucleotides of the invention comprise the second and third type 1 repeats of human TSP-1, the multimerization domain of human COMP, and the first type 2 repeat of human COMP. In another case, the chimeric proteins encoded by the polynucleotides of the invention are variants of the immediately aforementioned chimeric protein which have activity that is similar in quality and quantity to the anti-angiogenic activity of the protein whose amino acid sequence is represented in FIGS. 4A and 4B.

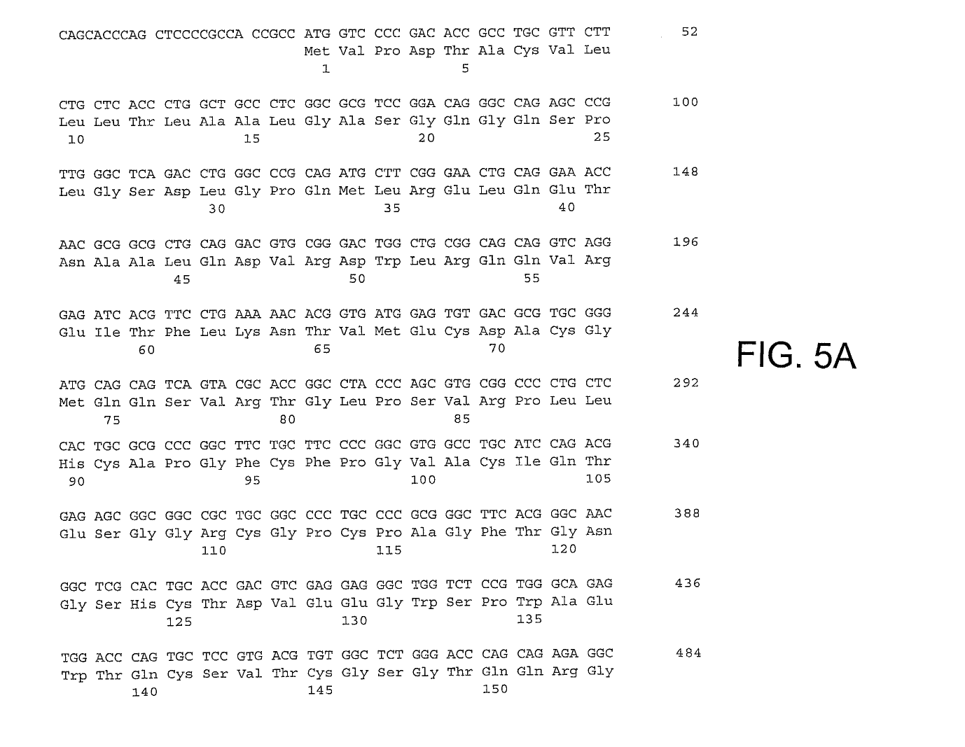

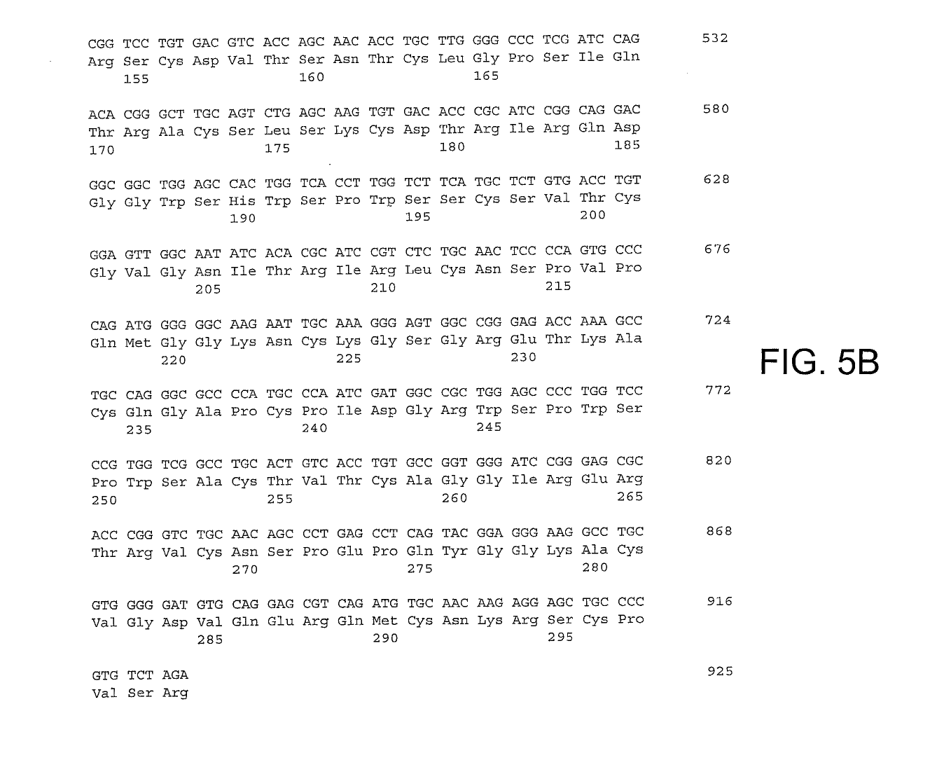

In one aspect, the invention comprises polynucleotides or nucleic acid molecules that encode chimeric proteins having portions whose amino acid sequences are derived from human TSP-2. The genomic structure of the human TSP-2 gene, which would provide one way to define the boundaries of the repeats, has not been determined. In one case, the chimeric protein encoded by the polynucleotides of the invention comprises the three type 1 repeats of human TSP-2. In another case, the chimeric proteins encoded by the polynucleotides of the invention are variants of the immediately aforementioned chimeric proteins which have activity that is similar in quality and quantity to the anti-angiogenic activity of the protein whose amino acid sequence is represented in FIGS. 5A and 5B. In another case, the chimeric protein encoded by polynucleotides of the invention comprises the three type 1 repeats of human TSP-2, and the multimerization domain of human COMP. In another case, the chimeric proteins encoded by the polynucleotides of the invention are variants of the immediately aforementioned chimeric protein which have activity that is similar in quality and quantity to the anti-angiogenic activity of the protein whose amino acid sequence is represented in FIGS. 5A and 5B.

The polynucleotides of the invention can be made by recombinant methods, can be made synthetically, can be replicated by enzymes in in vitro (e.g., PCR) or in vivo systems (e.g., by suitable host cells, when inserted into a vector appropriate for replication within the host cells), or can be made by a combination of methods. The polynucleotides of the invention can include DNA and its RNA counterpart.

As used herein, “nucleic acid,” “nucleic acid molecule,” “oligonucleotide” and “polynucleotide” include DNA and RNA and chemical derivatives thereof, including phosphorothioate derivatives and RNA and DNA molecules having a radioactive isotope or a chemical adduct such as a fluorophore, chromophore or biotin (which can be referred to as a “label”). The RNA counterpart of a DNA is a polymer of ribonucleotide units, wherein the nucleotide sequence can be depicted as having the base U (uracil) at sites within a molecule where DNA has the base T (thymidine).

Isolated nucleic acid molecules or polynucleotides can be purified from a natural source or can be made recombinantly. Polynucleotides referred to herein as “isolated” are polynucleotides purified to a state beyond that in which they exist in cells. They include polynucleotides obtained by methods described herein, similar methods or other suitable methods, and also include essentially pure polynucleotides produced by chemical synthesis or by combinations of biological and chemical methods, and recombinant polynucleotides that have been isolated. The term “isolated” as used herein for nucleic acid molecules, indicates that the molecule in question exists in a physical milieu distinct from that in which it occurs in nature. For example, an isolated polynucleotide may be substantially isolated with respect to the complex cellular milieu in which it naturally occurs, and may even be purified essentially to homogeneity, for example as determined by agarose or polyacrylamide gel electorphoresis or by A₂₆₀/A₂₈₀ measurements, but may also have further cofactors or molecular stabilizers (for instance, buffers or salts) added.

The invention further comprises the polypeptides encoded by the isolated nucleic acid molecules of the invention. Thus, for example, the invention relates to fusion proteins, comprising a portion of TSP-1 which comprises the second and third type 1 repeats, linked to a second moiety not occurring in TSP-1 as found in nature. In an analogous manner, the invention relates also to fusion proteins, comprising TSP-2 or a functional portion thereof such as one or more repeat regions as a first moiety, linked to second moiety not occurring in TSP-2 as found in nature. The second moiety can be an amino acid, peptide or polypeptide, and can have enzymatic or binding activity of its own. The first moiety can be in an N-terminal location, C-terminal location or internal to the fusion protein. In one embodiment, the fusion protein comprises the portion of human TSP-1 described immediately above, or human TSP-2 or a portion thereof as the first moiety, and a second moiety comprising a linker sequence and an affinity ligand.

Another aspect of the invention relates to a method of producing a chimeric protein of the invention, or a variant thereof, and to expression systems and host cells containing a vector appropriate for expression of a chimeric protein of the invention. Variants of the chimeric protein include those having amino acid sequences that differ from those sequences in FIGS. 4A and 4B, and FIGS. 5A and 5B, wherein those variants have several, such as 5 to 10, 1 to 5, or 3, 2 or 1 amino acids substituted, deleted, or added, in any combination, compared to the sequences in FIGS. 4A and 4B and FIGS. 5A and 5B. In one embodiment, variants have silent substitutions, additions and deletions that do not alter the properties and activities of the chimeric protein. Variants can also be modified polypeptides in which one or more amino acid residues are modified, and mutants comprising one or more modified residues.

Proteins and polypeptides described herein can be assessed for their angiogenic activity by using an assay such as those described in Tolsma, S. S. et al., J. Cell Biol. 122(2):497-511 (1993), one which measures the migration of bovine adrenal capillary endothelial cells in culture, and one which tests migration of cells into a sponge containing an agent to be tested for activity. A further test for angiogenesis, which can also be adapted also to test anti-angiogenesis activity, is described in Polverini, P. J. et al., Methods. Enzymol. 198:440-450 (1991).

Cells that express such a chimeric protein or a variant thereof can be made and maintained in culture, under conditions suitable for expression, to produce protein for isolation. These cells can be procaryotic or eucaryotic. Examples of procaryotic cells that can be used for expression (as “host cells”; “cell” including herein cells of tissues, cell cultures, cell strains and cell lines) include Escherichia coli, Bacillus subtilis and other bacteria. Examples of eucaryotic cells that can be used for expression include yeasts such as Saccharomyces cerevisiae, Schizosaccharomyces pombe, Pichia pastoris and other lower eucaryotic cells, and cells of higher eucaryotes such as those from insects and mammals. Suitable cells of mammalian origin include primary cells, and cell lines such as CHO, HeLa, 3T3, BHK, COS, 293, and Jurkat cells. Suitable cells of insect origin include primary cells, and cell lines such as SF9 and High five cells. (See, e.g., Ausubel, F. M. et al., eds. Current Protocols in Molecular Biology, Greene Publishing Associates and John Wiley & Sons Inc., (containing Supplements up through 1998)).

In one embodiment, host cells that produce a recombinant chimeric protein, variant, or portions thereof can be made as follows. A gene encoding a chimeric protein described herein can be inserted into a nucleic acid vector, e.g., a DNA vector, such as a plasmid, virus or other suitable replicon (including vectors suitable for use in gene therapy, such as those derived from adenovirus or others; see, for example Xu, M. et al., Molecular Genetics and Metabolism 63:103-109, 1998) can be present in a single copy or multiple copies, or the gene can be integrated in a host cell chromosome. A suitable replicon or integrated gene can contain all or part of the coding sequence for the protein or variant, operably linked to one or more expression control regions whereby the coding sequence is under the control of transcription signals and linked to appropriate translation signals to permit translation. The vector can be introduced into cells by a method appropriate to the type of host cells (e.g., transformation, electroporation, infection). For expression from the gene, the host cells can be maintained under appropriate conditions (e.g., in the presence of inducer, normal growth conditions, etc.). Proteins or polypeptides thus produced can be recovered (e.g., from the cells, the periplasmic space, culture medium) using suitable techniques.

The invention also relates to isolated proteins or polypeptides encoded by nucleic acids of the present invention. Isolated proteins can be purified from a natural source or can be made recombinantly. Proteins or polypeptides referred to herein as “isolated” are proteins or polypeptides purified to a state beyond that in which they exist in cells and include proteins or polypeptides obtained by methods described herein, similar methods or other suitable methods, and also include essentially pure proteins or polypeptides, proteins or polypeptides produced by chemical synthesis or by combinations of biological and chemical methods, and recombinant proteins or polypeptides which are isolated. Thus, the term “isolated” as used herein, indicates that the polypeptide in question exists in a physical milieu distinct from the cell in which its biosynthesis occurs. For example, an isolated COMP/TSP-1 or COMP/TSP-2 chimeric protein may be purified essentially to homogeneity, for example as determined by PAGE or column chromatography (for example, HPLC), but may also have further cofactors or molecular stabilizers added to the purified protein to enhance activity. In one embodiment, proteins or polypeptides are isolated to a state at least about 75% pure; more preferably at least about 85% pure, and still more preferably at least about 95% pure, as determined by Coomassie blue staining of proteins on SDS-polyacrylamide gels.

Chimeric or fusion proteins can be produced by a variety of methods. For example, a chimeric protein can be produced by the insertion of a TSP gene or portion thereof into a suitable expression vector, such as Bluescript SK +/−(Stratagene), pGEX-4T-2 (Pharmacia), pET-15b, pET-20b(+) or pET−24(+) (Novagen). The resulting construct can be introduced into a suitable host cell for expression. Upon expression, chimeric protein can be purified from a cell lysate by means of a suitable affinity matrix (see e.g., Current Protocols in Molecular Biology (Ausubel, F. M. et al., eds., Vol. 2, pp. 16.4.1-16.7.8, containing supplements up through Supplement 44, 1998).

Polypeptides of the invention can be recovered and purified from cell cultures by well-known methods. The recombinant protein can be purified by ammonium sulfate precipitation, heparin-Sepharose affinity chromatography, gel filtration chromatography and/or sucrose gradient ultracentrifugation using standard techniques. Further methods that can be used for purification of the polypeptide include ethanol precipitation, acid extraction, anion or cation exchange chromatography, phosphocellulose chromatography, hydrophobic interaction chromatography, affinity chromatography, hydroxylapatite chromatography and high performance liquid chromatography. Known methods for refolding protein can be used to regenerate active conformation if the polypeptide is denatured during isolation or purification.

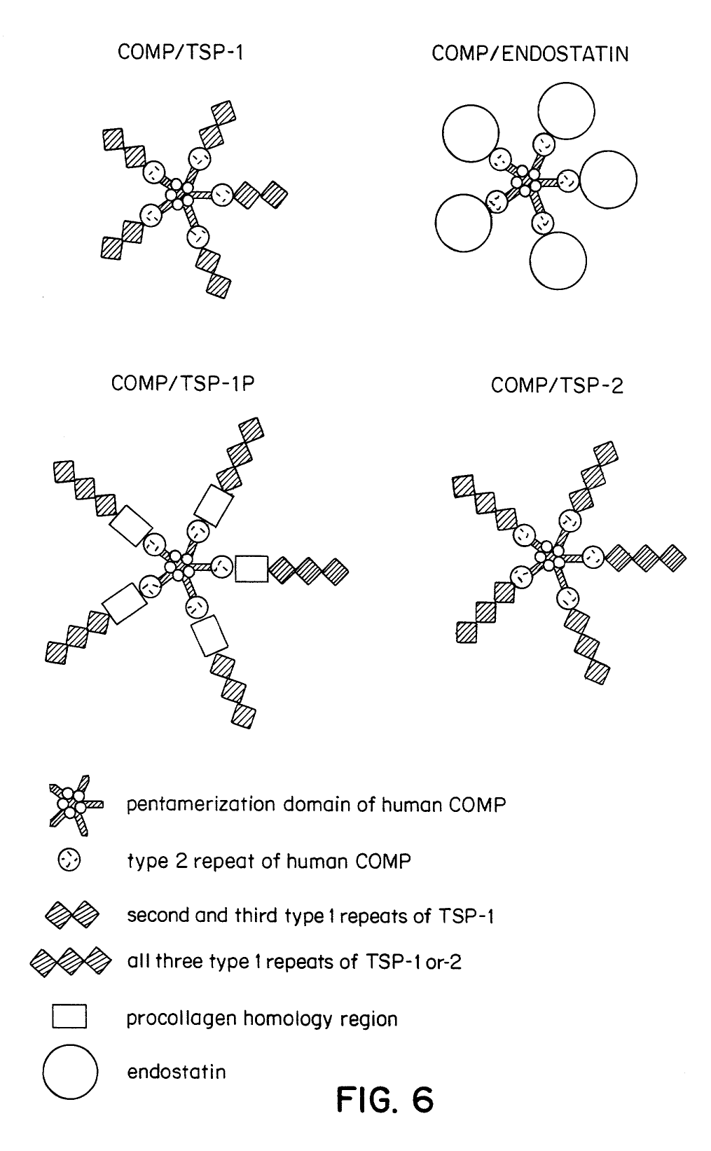

The method to construct genes encoding COMP/TSP-1 or COMP/TSP-2 hybrid proteins can be applied more broadly to produce polynucleotides, and vectors and host cells comprising such polynucleotides, wherein the polynucleotides encode COMP/endostatin, COMP/angiostatin, COMP/platelet factor 4, or COMP/prolactin, for example. In each case, a portion of a polynucleotide known to encode full-length human endostatin, angiostatin, platelet factor 4 (GenBank Accession No. M25897) or prolactin (GenBank Accession No. V00566), can be chosen for cloning into a COMP cDNA as illustrated herein for COMP/TSP-1 and COMP/TSP-2 DNA constructs. Thus, the invention also includes COMP/endostatin, COMP/angiostatin, COMP/platelet factor 4, and COMP/prolactin chimeric proteins encoded by such nucleic acid constructs. See FIG. 6 for a schematic representation of the structure of COMP/endostatin.

In addition, a portion of the endostatin, angiostatin, platelet factor 4 or prolactin coding regions, wherein that portion encodes a polypeptide having anti-angiogenic activity, can be added to or incorporated into a DNA construct encoding COMP/TSP-1, such that a TSP-1-derived polypeptide and a polypeptide derived from endostatin, angiostatin, platelet factor 4 or prolactin are produced fused together in tandem on the same “arm” of the “5-armed” COMP-multimerized pentamer. Different expression constructs can be introduced into the same host cells such that two or more chimeric protein “arms” of different types (e.g., COMP/angiostatin and COMP/TSP-1 or COMP/TSP-2) are joined at the COMP multimerization domain.

Chimeric protein antiangiogenic agents can be used, for example, after surgery or radiation to prevent recurrence of metastases, in combination with conventional chemotherapy, immunotherapy, or various types of gene therapy not necessarily directed against angiogenesis.

Construction of COMP/TSP-1P Expression Vectors

Expression vectors that can be used to produce COMP/TSP-1P, a chimeric protein that includes the procollagen homology region (see FIG. 6), can be produced from two distinct cDNAs. The COMP portion is identical to that in the Examples described herein. For TSP-1, a new forward primer (GAT GAC GTC ACT GAA GAG AAC AAA GAG) (SEQ ID NO: 14) and the same reverse primer as described in the Examples can be used to produce a PCR product that is approximately 750 base pairs in size and has an AatII restriction endonuclease site at the 5′ end and an XbaI restriction endonuclease site at the 3′ end. The product codes for amino acids 284-530 and includes the procollagen homology region (exons 6 and 7) and type 1 repeats. If inclusion of the TGF-β activating sequence (RFK) that is in the first type 1 repeat is found to reduce the antitumor activity, this sequence will be mutated to an inactive sequence (QFK, for example) using an oligonucleotide-directed mutagenesis kit (Amersham). The COMP/TSP-1P expression vector can be constructed by cutting the PCR product with AatII and XbaI and cloning it into the COMP cDNA cut with the same enzymes. The protein can be expressed using the methods that have been described for COMP/TSP-1 and COMP/TSP-2.

Construction of COMP/Endostatin Expression Vectors

The strategy for making multimers of the TSP-1 and TSP-2 can be used to make multimers of other anti-angiogenic proteins. For example, if the active region of endostatin is prepared by PCR and cloned into the COMP cDNA, a pentameric structure of endostatin can be made when this construct is expressed (O'Reilly M. D., et al., Cell 88:277-285, (1997)). In addition, if the COMP/TSP-1 and the COMP/endostatin genes are expressed concurrently within the same cells, mixed pentamers of COMP/TSP- 1 and COMP/endostatin subunits are made. The mixed multimer allows simultaneous treatment with the two reagents by delivery of a single therapeutic. An additive or synergistic effect of the two agents may significantly increase the efficacy of this reagent as compared to that of each reagent alone. For example, combination therapy with angiostatin and endostatin has eradicated tumors in mice (Boehm, T. et al., Nature 390:404-407, 1997).

The cDNA for endostatin can be prepared by PCR of liver cDNA or from an isolated cDNA clone for collagen XVIII (GenBank accession no. L22548). The human endostatin cDNA can be produced by PCR with the forward primer GAT GAC GTC CAC AGC CAC CGC G (SEQ ID NO: 15) and the reverse primer GAT TCT AGA CTA CTT GGA GGC AGT CAT G (SEQ ID NO: 16). The resulting PCR product is approximately 560 base pairs and encodes amino acids 1 to 184 of human endostatin (Sasaki, T., et al., EMBO J., 17:4249-4256, 1998). The COMP/endostatin expression vector can be constructed by cutting the PCR product with AatII and XbaI, and cloning it into cDNA cut with the same enzymes. The protein can be expressed using the methods that have been described herein for COMP/TSP-1 and COMP/TSP-2. Angiostatin, as it was isolated from mice bearing Lewis lung carcinoma, includes the first four kringle domains of plasminogen (amino acids 98-440) (O'Reilly, M. S., et al., Cell 79:315-328, 1994). It should be noted that smaller constructs that contain fewer kringle domains should also be active based on published data (Griscelli, F., et al., Proc. Natl. Acad. Sci. USA 95:6367-6372, 1998). A 16,000 dalton fragment of prolactin and platelet factor 4 have also been reported to inhibit angiogenesis (Clapp, C. et al., Endocrinology 133:1292-1299, 1993; Gapta, S. K., et al., Proc. Natl. Acad. Sci. USA 92:7799-7803, 1995).

Also included in the inventions are compositions containing, as a biological ingredient, an anti-angiogenic chimeric protein, or a variant thereof to inhibit angiogenesis in mammalian tissues, and use of such compositions in the treatment of diseases and conditions characterized by, or associated with, angiogenic activity. Such methods can involve administration by oral, topical, injection, implantation, sustained release, or other delivery methods that bring one or more anti-angiogenic chimeric proteins in contact with cells whose growth is to be inhibited.

The present invention includes a method of treating an angiogenesis-mediated disease with a therapeutically effective amount of one or more anti-angiogenic chimeric proteins. Angiogenesis-mediated diseases can include, but are not limited to, cancers, solid tumors, tumor metastasis, benign tumors (e.g., hemangiomas, acoustic neuromas, neurofibromas, trachomas, and pyogenic granulomas), rheumatoid arthritis, psoriasis, ocular angiogenic diseases (e.g., diabetic retinopathy, retinopathy of prematurity, macular degeneration, corneal graft rejection, neovascular glaucoma, retrolental fibroplasia, rubeosis), Osler-Webber Syndrome, myocardial angiogenesis, plaque neovascularization, telangiectasia, hemophiliac joints, angiofibroma, and wound granulation.

“Cancer” means neoplastic growth, hyperplastic or proliferative growth or a pathological state of abnormal cellular development and includes solid tumors, non-solid tumors, and any abnormal cellular proliferation, such as that seen in leukemia. As used herein, “cancer” also means angiogenesis-dependent cancers and tumors, i.e., tumors that require for their growth (expansion in volume and/or mass) an increase in the number and density of the blood vessels supplying them with blood. “Regression” refers to the reduction of tumor mass and size. As used herein, the term “therapeutically effective amount” means the total amount of each active component of the composition or method that is sufficient to show a meaningful benefit to a treated human or other mammal, i.e., treatment, healing, prevention or amelioration of the relevant medical condition, or an increase in rate of treatment, healing, prevention or amelioration of such conditions. More specifically, for example, a therapeutically effective amount of an anti-angiogenic chimeric protein can cause a measurable reduction in the size or numbers of tumors, or in their rate of growth or multiplication, compared to untreated tumors. Other methods of assessing a “therapeutically effective amount,” can include the result that blood vessel formation is measurably reduced in treated tissues compared to untreated tissues.

One or more anti-angiogenic chimeric proteins may be used in combination with other compositions and procedures for the treatment of diseases. For example, a tumor may be treated conventionally with surgery, radiation, chemotherapy, or immunotherapy, combined with anti-angiogenic chimeric proteins, and then anti-angiogenic chimeric proteins may be subsequently administered to the patient to extend the dormancy of micrometastases and to stabilize and inhibit the growth of any residual primary tumor.

The compositions may further contain other agents which either enhance the activity of the protein or compliment its activity or use in treatment, such as chemotherapeutic or radioactive agents. Such additional factors and/or agents may be included in the composition to produce a synergistic effect with protein of the invention, or to minimize side effects. Additionally, administration of the composition of the present invention may be administered concurrently with other therapies, e.g., administered in conjunction with a chemotherapy, immunotherapy or radiation therapy regimen.

The angiogenesis-modulating composition of the present invention may be a solid, liquid or aerosol and may be administered by any known route of administration. Examples of solid compositions include pills, creams, and implantable dosage units. The pills may be administered orally, the therapeutic creams may be administered topically. The implantable dosage unit may be administered locally, for example at a tumor site, or may be implanted for systemic release of the angiogenesis-modulating composition, for example subcutaneously. Examples of liquid composition include formulations adapted for injection subcutaneously, intravenously, intraarterially, and formulations for topical and intraocular administration. Examples of aerosol formulation include inhaler formulation for administration to the lungs.

The anti-angiogenic chimeric proteins can be provided as isolated and substantially purified proteins in pharmaceutically acceptable formulations (including aqueous or nonaqueous carriers or solvents) using formulation methods known to those of ordinary skill in the art. These formulations can be administered by standard routes. In general, the combinations may be administered by the topical, transdermal, intraperitoneal, intracranial, intracerebroventricular, intracerebral, intravaginal, intrauterine, oral, rectal or parenteral (e.g., intravenous, intraspinal, subcutaneous or intramuscular) route. In addition, the anti-angiogenic chimeric proteins may be incorporated into biodegradable polymers allowing for sustained release of the compound, the polymers being implanted in the vicinity of where drug delivery is desired, for example, at the site of a tumor, or implanted so that the anti-angiogenic chimeric proteins is slowly released systemically. Osmotic minipumps may also be used to provide controlled delivery of high concentrations of anti-angiogenic chimeric proteins through cannulae to the site of interest, such as directly into a growth or into the vascular supply to that growth. The biodegradable polymers and their use are described, for example, in detail in Brem et al (1991) (J. Neurosurg. 74:441-446), which is hereby incorporated by reference in its entirety.

As used herein, the terms “pharmaceutically acceptable,” as it refers to compositions, carriers, diluents and reagents, represents that the materials are capable of administration to or upon a mammal with a minimum of undesirable physiological effects such as nausea, dizziness, gastric upset and the like. The preparation of a pharmacological composition that contains active ingredients dissolved or dispersed therein is well understood in the art and need not be limited based on formulation. Typically, such compositions are prepared as injectables either as liquid solutions or suspensions, however, solid forms suitable for solution, or suspensions, in liquid prior to use can also be prepared. The preparation can also be emulsified, for example, in liposomes.

The dosage of the anti-angiogenic chimeric proteins of the present invention will depend on the disease state or condition being treated and other clinical factors such as weight and condition of the human or animal and the route of administration of the compound. It is to be understood that the present invention has application for both human and veterinary use. The methods of the present invention contemplate single as well as multiple administrations, given either simultaneously or over an extended period of time.

The present invention also encompasses gene therapy whereby a polynucleotide encoding one or more anti-angiogenic chimeric proteins or one or more variants thereof, is introduced and regulated in a patient. Various methods of transferring or delivering DNA to cells for expression of the gene product protein, otherwise referred to as gene therapy, are disclosed in Gene Transfer into Mammalian Somatic Cells in Vivo, N. Yang (1992) Crit. Rev. Biotechnol. 12(4):335-356, which is hereby incorporated by reference. Gene therapy encompasses incorporation of DNA sequences into somatic cells or germ line cells for use in either ex vivo or in vivo therapy. Gene therapy can function to replace genes, augment normal or abnormal gene function, and to combat infectious diseases and other pathologies.

Strategies for treating these medical problems with gene therapy include therapeutic strategies such as identifying the defective gene and then adding a functional gene to either replace the function of the defective gene or to augment a slightly functional gene; or prophylactic strategies, such as adding a gene for the product protein that will treat the condition or that will make the tissue or organ more susceptible to a treatment regimen. For example, a gene encoding an anti-angiogenic chimeric protein may be inserted into tumor cells of a patient and thus inhibit angiogenesis.

Gene transfer methods for gene therapy fall into three broad categories: physical (e.g., electroporation, direct gene transfer and particle bombardment), chemical (e.g., lipid-based carriers, or other non-viral vectors) and biological (e.g., virus-derived vector and receptor uptake). For example, non-viral vectors may be used which include liposomes coated with DNA. Such liposome/DNA complexes may be directly injected intravenously into the patient. It is believed that the liposome/DNA complexes are concentrated in the liver where they deliver the DNA to macrophages and Kupffer cells. These cells are long lived and thus provide long term expression of the delivered DNA. Additionally, vectors or the “naked” DNA of the gene may be directly injected into the desired organ, tissue or tumor for targeted delivery of the therapeutic DNA.

In vivo gene transfer involves introducing the DNA into the cells of the patient when the cells are within the patient. Methods include using virally mediated gene transfer using a noninfectious virus to deliver the gene in the patient or injecting naked DNA into a site in the patient and the DNA is taken up by a percentage of cells in which the gene product protein is expressed. Additionally, the other methods described herein, such as use of a “gene gun,” may be used for in vitro insertion of anti-angiogenic chimeric proteins DNA or anti-angiogenic chimeric proteins regulatory sequences.

Chemical methods of gene therapy may involve a lipid based compound, not necessarily a liposome, to transfer the DNA across the cell membrane. Lipofectins or cytofectins, lipid-based positive ions that bind to negatively charged DNA, make a complex that can cross the cell membrane and provide the DNA into the interior of the cell. Another chemical method uses receptor-based endocytosis, which involves binding a specific ligand to a cell surface receptor and enveloping and transporting it across the cell membrane. The ligand binds to the DNA and the whole complex is transported into the cell. The ligand gene complex is injected into the blood stream and then target cells that have the receptor will specifically bind the ligand and transport the ligand-DNA complex into the cell.

Many gene therapy methodologies employ viral vectors to insert genes into cells. For example, altered retrovirus vectors have been used in ex vivo methods to introduce genes into peripheral and tumor-infiltrating lymphocytes, hepatocytes, epidermal cells, myocytes, or other somatic cells. These altered cells are then introduced into the patient to provide the gene product from the inserted DNA.

Viral vectors have also been used to insert genes into cells using in vivo protocols. To direct the tissue-specific expression of foreign genes, cis-acting regulatory elements or promoters that are known to be tissue-specific can be used. Alternatively, this can be achieved using in situ delivery of DNA or viral vectors to specific anatomical sites in vivo. For example, gene transfer to blood vessels in vivo was achieved by implanting in vitro transduced endothelial cells in chosen sites on arterial walls. The virus infected surrounding cells which also expressed the gene product. A viral vector can be delivered directly to the in vivo site, by a catheter for example, thus allowing only certain areas to be infected by the virus, and providing long-term, site specific gene expression. In vivo gene transfer using retrovirus vectors has also been demonstrated in mammary tissue and hepatic tissue by injection of the altered virus into blood vessels leading to the organs.

Viral vectors that have been used for gene therapy protocols include but are not limited to, retroviruses, other RNA viruses such as poliovirus or Sindbis virus, adenovirus, adeno-associated virus, herpes viruses, SV40, vaccinia and other DNA viruses. Replication-defective murine retroviral vectors have been widely utilized gene transfer vectors.

Carrier mediated gene transfer in vivo can be used to transfect foreign DNA into cells. The carrier-DNA complex can be conveniently introduced into body fluids or the bloodstream and then site-specifically directed to the target organ or tissue in the body. Both liposomes and polycations, such as polylysine, lipofectins or cytofectins, can be used. Liposomes can be developed which are cell specific or organ specific and thus the foreign DNA carried by the liposome will be taken up by target cells. Injection of immunoliposomes that are targeted to a specific receptor on certain cells can be used as a convenient method of inserting the DNA into the cells bearing the receptor. Another carrier system that has been used is the asialoglycoprotein/polylysine conjugate system for carrying DNA to hepatocytes for in vivo gene transfer.

The gene therapy protocol for transfecting anti-angiogenic chimeric proteins into a patient may either be through integration of a gene encoding an anti-angiogenic chimeric protein into the genome of the cells, into minichromosomes or as a separate replicating or non-replicating DNA construct in the cytoplasm or nucleoplasm of the cell. Anti-angiogenic chimeric proteins expression may continue for a long-period of time or may be reinjected periodically to maintain a desired level of the anti-angiogenic chimeric proteins protein in the cell, the tissue or organ or a determined blood level.

### EXAMPLES

### Example 1

### Construction of COMP/TSP-1 and COMP/TSP-2

The chimeric expression vectors have been produced from three distinct cDNAs. The first is a clone for human cartilage oligomeric matrix protein (COMP) and was isolated from a λgtll chondrocyte cDNA library (Doege, K. J, et al., J. Biol. Chem. 266:894-902 (1991)). This is an almost full-length clone for the COMP mRNA that only lacks a small region of the 5′-untranslated region. This clone (hCOMP-95) was used previously to determine the sequence of human COMP (GenBank Accession No. L32137; Genomics, 24:435-439 (1994)).

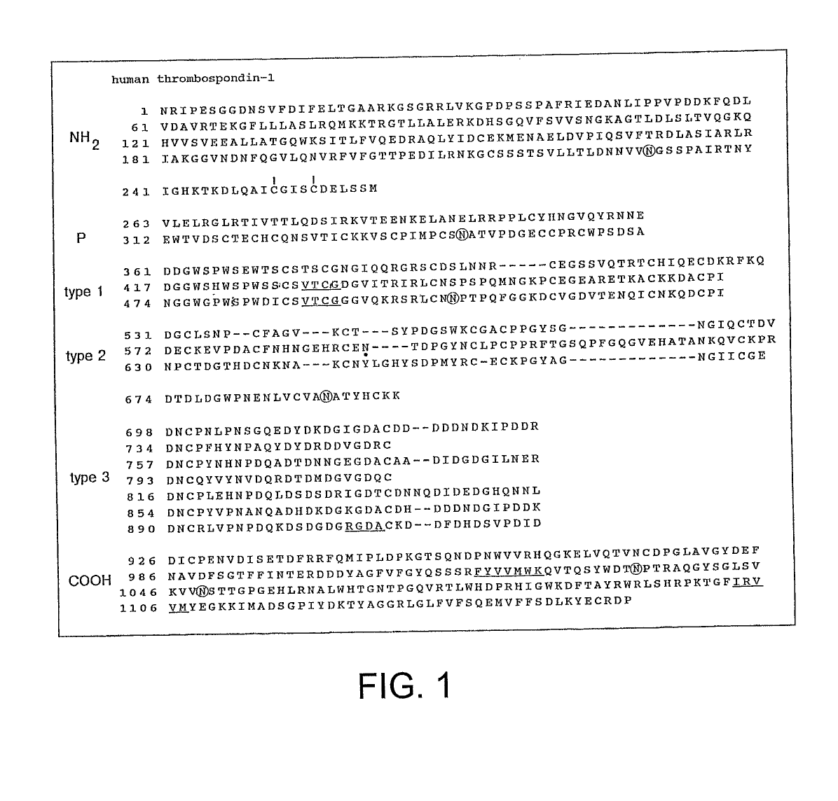

The second cDNA was produced using the polymerase chain reaction (PCR) with the human thrombospondin-1 (TSP-1) gene as the template. The TSP-1 clones were isolated from a human endothelial cell library (J. Cell Biol. 103:1635-1648 (1986)). The forward primer (GAT GAC GTC GAT GGT GGC TGG AGC CAC) (SEQ ID NO: 17) and the reverse primer (GAT CTA GAT TGG ACA GTC CTG CTT G) (SEQ ID NO: 18) produce a PCR product that is approximately 354 basepairs in size and has an Aat II restriction endonuclease site at the 5′ end and an Xba I restriction endonuclease site at the 3′ end. The PCR product encodes amino acids 417 to 530 and includes the second and third type 1 repeats of TSP-1 (see FIG. 1 for the numbering of amino acids in TSP-1). The coding sequence for the first type 1 repeat was not included in the PCR product, by design, because it contains an RFK sequence that has been shown to activate TGF-β. This activity is not required to inhibit angiogenesis and it may produce unwanted secondary effects on numerous cell types. Vectors that include the first type 1 repeat can be constructed, using the same approach, if this region is found to enhance the antiangiogenic activity or other activities.

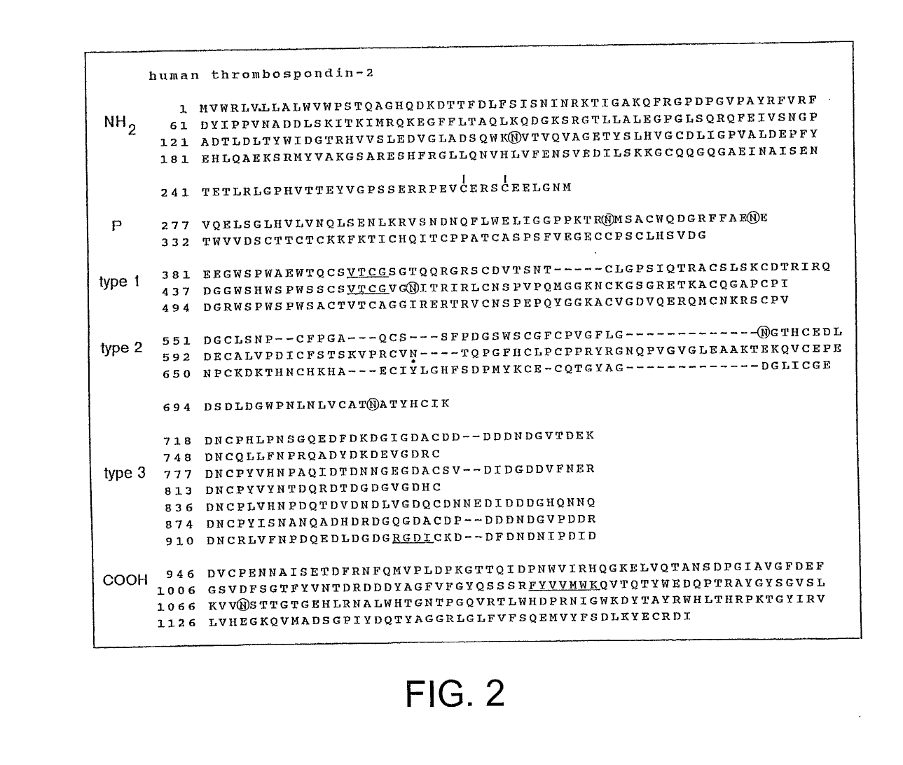

The third cDNA was produced by PCR with a human heart cDNA library (catalog no. 936208 from Stratagene, LaJolla, Calif.) as the template. The forward primer (GAT GAC GTC GAG GAG GGC TGG TCT CCG) (SEQ ID NO: 19) and the reverse primer (GAT CTA GAC ACG GGG CAG CTC CTC TTG) (SEQ ID NO: 20) produced a PCR product that is approximately 520 base pairs in size and has an Aat II restriction endonuclease site at the 5′ end and an Xba I restriction endonuclease site at the 3′ end. The PCR product codes for amino acids 381 to 550 of TSP-2 and, includes all three type 1 repeats of TSP-2 (see FIG. 2 for numbering of amino acids in TSP-2). The sequence of the PCR primers was based on the human TSP-2 sequence in the GenBank database (Accession No. L12350). The sequences of the PCR products were determined to establish that mutations that affect the amino acid sequence had not been introduced during the PCR.

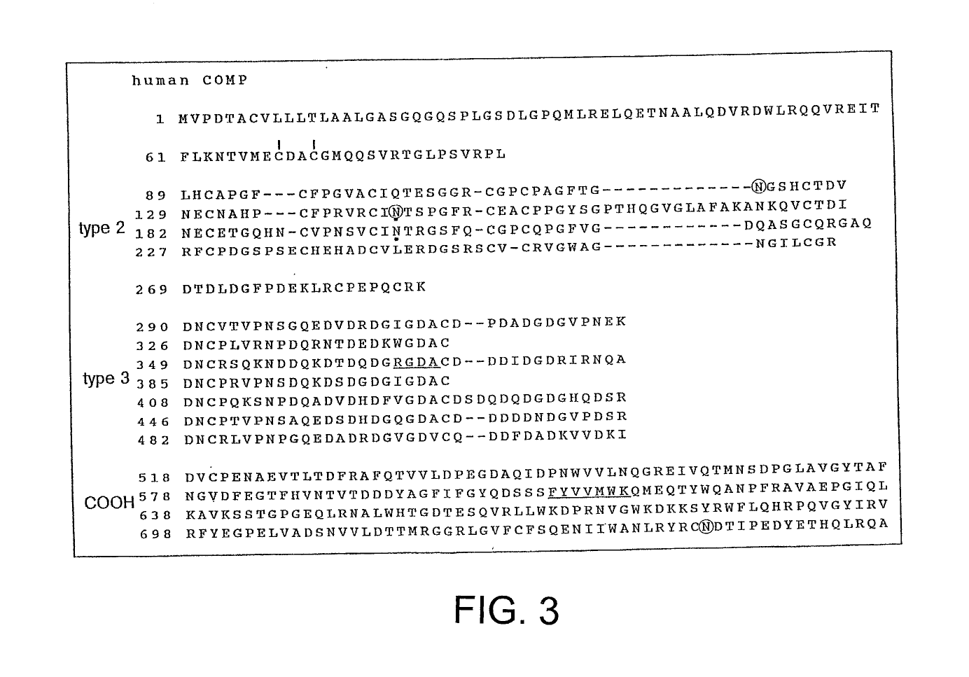

The COMP/TSP-1 and COMP-TSP-2 expression vectors were constructed by cutting the PCR products with Aat II and Xba I and subcloning them into the COMP cDNA vector [derived from Bluescript (Stratagene, La Jolla, Calif.)] cut with the same enzymes. The portion of COMP that was retained includes the signal sequence, the regions required for pentamerization and the first type 2 repeat (amino acids 1 to 128 on the enclosed sequence; FIG. 3). Since there was an internal Aat II site in the TSP-2 PCR product, it had to be cloned into the vector in two steps. A 430 basepair Aat II/Xba I fragment of the TSP-2 PCR product was subcloned into the vector containing the portion of COMP as a first step. The resulting subclone was cut with Aat II, and a 90 base pair Aat II fragment of the PCR product was ligated into the expression vector. The final forms of the cDNAs were confirmed to have the predicted structure by nucleotide sequencing. They were then cut with Eco R1 and Xba I and ligated into the pcDNA 3.1 (Invitrogen; Carlsbad, Calif.) vector cut with the same enzymes. The DNA sequences of COMP/TSP- 1 and COMP/TSP-2 are shown in FIGS. 4A and 4B and FIGS. 5A and 5B, respectively. The predicted molecular weights of the subunits of COMP/TSP-1 and COMP/TSP-2 should be approximately 24,200 and 30,000, respectively. The fully assembled COMP/TSP-1 and COMP/TSP-2 proteins should be 121,000 Da and 150,000 Da, respectively. The amino acid sequences of these proteins are shown in FIGS. 4A and 4B and FIGS. 5A and 5B, respectively.

### Example 2

### Production of Isolated COMP/TSP-1 and COMP/TSP-2

To express these chimeric proteins, the expression vectors can be transfected into human kidney 293 cells using the Lipofectin protocol (Gibco Laboratories). The cells can be selected with Zeocin and individual clones can be grown. The secretion of COMP/TSP-1 and COMP/TSP-2 can be monitored with western blotting using polyclonal antibodies to the region of COMP that is present in both expressed proteins. These antibodies have been produced by immunizing rabbits with a synthetically produced peptide, having an amino acid sequence derived from the N-terminal end of COMP, linked to a carrier protein. The amino acid sequence of the peptide is: SDLGPQMLRELQETN (SEQ ID NO: 21). A clone that expresses high levels of the protein can be grown in large volume flasks and in serum free media.

### Example 3

### Inhibition of Tumor Growth by COMP/TSP-1

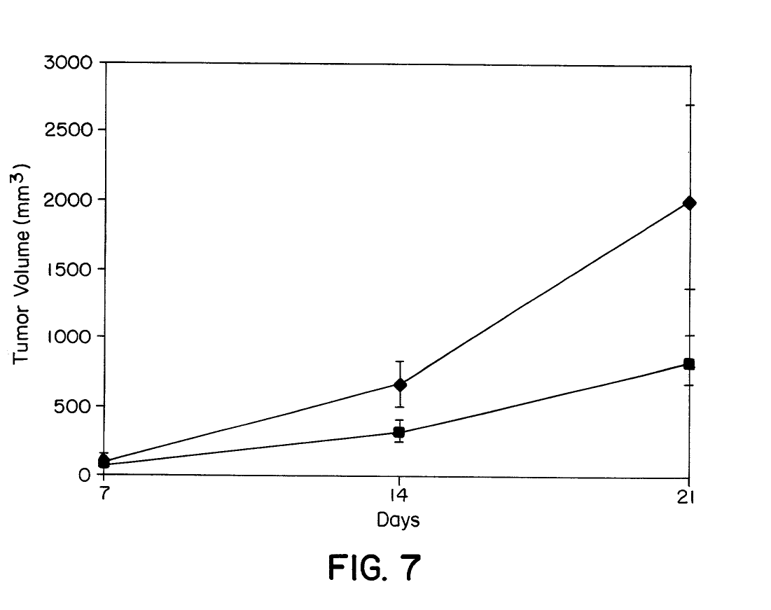

A cDNA of thrombospondin-1 (TSP-1) containing the second and third type-1 repeats and the COMP assembly sequence (COMP/TSP-1) was produced by PCR using constructs derived as above as template, and was cloned into the expression vector pNeo (Invitrogen, Carlsbad, Calif.). Both the resulting COMP/TSP-1 construct and the unaltered vector alone were transfected into the human squamous carcinoma cell line A431 (Streit, M., et al., American Journal of Pathology 155:441-452, 1999), and positive clones were selected using Geneticin at a concentration of 800 μg/ml. The growth curves of positive clones were determined over an 8 day period. Clones of pNeo- and COMP/TSP-1 construct-transfected cells that had similar growth curves were selected to test the effect of the chimeric protein on tumor growth in nude mice. A total of five mice pre group were injected intradermally at the shoulders with 5×10⁶ cells per site, two sites per mouse. Every week the tumors were measured with calipers. At three weeks, the mice were sacrificed and the tumors were removed for further studies. As can be seen from FIG. 7, expression of COMP/TSP-1 caused inhibition of the growth of the tumors in this model.

All references (e.g., journal articles, books, published patent applications and patents, etc.) cited herein are hereby incorporated by reference.

While this invention has been particularly shown and described with references to preferred embodiments thereof, it will be understood by those skilled in the art that various changes in form and details may be made therein without departing from the spirit and scope of the invention as defined by the appended claims.

What is claimed is:

1. An isolated nucleic acid molecule encoding a chimeric protein comprising the second and third type 1 repeats of human TSP-1, but not the TGF-β activation region of human TSP-1.

2. An isolated nucleic acid molecule encoding a chimeric protein comprising the multimerization domain of human COMP, the first type 2 repeat of human COMP, and the second and third type 1 repeats of human TSP-1.

3. An isolated nucleic acid molecule encoding a chimeric protein comprising the multimerization domain of human COMP, the first type 2 repeat of human COMP, and the second and third type 1 repeats of human TSP-1, but not the TGF-β activation region of human TSP-1.

4. An isolated nucleic acid molecule encoding a chimeric protein comprising the multimerization domain of human COMP, the procollagen homology region of TSP-1, and the first, second, and third type 1 repeats of human TSP-1.

5. An isolated nucleic acid molecule encoding a chimeric protein comprising the multimerization domain of human COMP, the procollagen homology region of TSP-1, and the first, second, and third type 1 repeats of human TSP-1, but not the TGF-β activation region of human TSP-1.

6. An isolated nucleic acid molecule encoding a chimeric protein comprising the multimerization domain of human COMP and a polypeptide derived from human endostatin having anti-angiogenic activity, wherein the chimeric protein has anti-angiogenic activity.

7. An isolated nucleic acid molecule encoding a chimeric protein comprising the multimerization domain of human COMP and a polypeptide derived from human angiostatin having anti-angiogenic activity, wherein the chimeric protein has anti-angiogenic activity.

8. An isolated nucleic acid molecule encoding a chimeric protein comprising the multimerization domain of human COMP and a polypeptide derived from human prolactin having anti-angiogenic activity, wherein the chimeric protein has anti-angiogenic activity.

9. An isolated nucleic acid molecule encoding a chimeric protein comprising the multimerization domain of human COMP and a polypeptide derived from a portion of human platelet factor 4 having anti-angiogenic activity, wherein the chimeric protein has anti-angiogenic activity.

10. An isolated nucleic acid molecule encoding a chimeric protein comprising the multimerization domain of human COMP, the procollagen homology region of human TSP-2, and the first, second and third type 1 repeats of human TSP-2.

11. An isolated nucleic acid molecule encoding a protein having the amino acid sequence SEQ ID NO: 5.

12. A vector comprising nucleic acid encoding a chimeric protein comprising the second and third type 1 repeats of human TSP-1 but not the TGF-β activation region of human TSP-1.

13. A host cell comprising the vector of claim 12.

14. A vector comprising nucleic acid encoding a chimeric protein comprising the multimerization domain of human COMP, the first type 2 repeat of human COMP, and the second and third type 1 repeats of human TSP-1.

15. A host cell comprising the vector of claim 14.

16. A method for producing a chimeric protein which comprises the multimerization domain of human COMP, the first type 2 repeat of human COMP, and the second and third type 1 repeats of human TSP-1, said method comprising maintaining the host cell of claim 15 under conditions suitable for expression of said nucleic acid, whereby said protein is produced.

17. The method of claim 16 further comprising isolating the chimeric protein.

18. A vector comprising nucleic acid encoding a chimeric protein comprising the multimerization domain of human COMP, the first type 2 repeat of human COMP, and the second and third type 1 repeats of human TSP-1, but not the TGF-β activation region of human TSP-1.

19. A host cell comprising the vector of claim 18.

20. A method for producing a chimeric protein which comprises the multimerization domain of human COMP, the first type 2 repeat of human COMP, and the second and third type 1 repeats of human TSP-1, but not the TGF-β activation region of human TSP-1, said method comprising maintaining the host cell of claim 19 under conditions suitable for expression of said nucleic acid, whereby said protein is produced.

21. The method of claim 20 further comprising isolating the chimeric protein.

22. A vector comprising nucleic acid encoding a chimeric protein comprising the multimerization domain of human COMP, the procollagen homology region, and the first, second, and third type 1 repeats of human TSP-1.

23. A vector comprising nucleic acid encoding a protein having the amino acid sequence SEQ ID NO: 5.

24. A host cell comprising the vector of claim 23.

25. A chimeric protein comprising the second and third type 1 repeat of human TSP-1 but not the TGF-β activation region of human TSP-1.

26. A chimeric protein comprising the multimerization domain of human COMP, the first type 2 repeat of human COMP, and the second and third type 1 repeats of human TSP-1.

27. A chimeric protein comprising the multimerization domain of human COMP, the first type 2 repeat of human COMP, and the second and third type 1 repeats of human TSP-1, but not the TGF-β activation region of human TSP-1.

28. A chimeric protein comprising the multimerization domain of human COMP, the procollagen homology region of TSP-1, and the first, second, and third type 1 repeats of human TSP-1.

29. A chimeric protein comprising the multimerization domain of human COMP and a portion of human endostatin, wherein the chimeric protein has anti-angiogenic activity.

30. A chimeric protein comprising the multimerization domain of human COMP and a portion of human angiostatin, wherein the chimeric protein has anti-angiogenic activity.

31. A chimeric protein comprising the multimerization domain of human COMP and a portion of human prolactin, wherein the chimeric protein has anti-angiogenic activity.

32. A chimeric protein comprising the multimerization domain of human COMP and a portion of human platelet factor 4, wherein the chimeric protein has anti-angiogenic activity.

33. A protein having the amino acid sequence SEQ ID NO: 5.

34. An isolated nucleic acid molecule encoding a chimeric protein comprising the three type 1 repeats of human TSP-2.

35. A vector comprising nucleic acid encoding a chimeric protein comprising the three type 1 repeats of human TSP-2.

36. A host cell comprising the vector of claim 35.

37. A method for producing a chimeric protein which comprises the three type 1 repeats of human TSP-2, said method comprising maintaining the host cell of claim 36 under conditions suitable for expression of said nucleic acid, whereby said protein is produced.

38. The method of claim 37 further comprising isolating the chimeric protein.

39. A isolated nucleic acid molecule encoding a chimeric protein comprising the multimerization domain of human COMP, the first type 2 repeat of human COMP, and the three type 1 repeats of human TSP-2.

40. A vector comprising isolated nucleic acid encoding a chimeric protein comprising the multimerization domain of human COMP, the first type 2 repeat of human COMP, and the three type 1 repeats of human TSP-2.

41. A host cell comprising the vector of claim 40.

42. A method for producing a chimeric protein which comprises the multimerization domain of human COMP, the first type 2 repeat of human COMP, and the three type 1 repeats of human TSP-2, said method comprising maintaining the host cell of claim 41 under conditions suitable for expression of said nucleic acid, whereby said protein is produced.

43. The method of claim 42 further comprising isolating the chimeric protein.

44. A isolated nucleic acid molecule encoding a protein having the amino acid sequence SEQ ID NO: 7.

45. A vector comprising nucleic acid encoding a protein having the amino acid sequence SEQ ID NO: 7.

46. A host cell comprising the vector of claim 45.

47. A chimeric protein comprising the three type 1 repeats of human TSP-2.

48. A chimeric protein comprising the procollagen homology region of TSP-2 and the three type 1 repeats of human TSP-2.

49. A chimeric protein comprising the multimerization domain of human COMP, the first type 2 repeat of human COMP, and the three type 1 repeats of human TSP-2.

50. A protein having the amino acid sequence SEQ ID NO: 7.

51. A method for inhibiting angiogenesis in a human or other mammal, the method comprising administering to the human or other mammal a therapeutically effective amount of an anti-angiogenic chimeric protein.

52. The method of claim 51 wherein the anti-angiogenic chimeric protein is selected from the group consisting of:
a) a chimeric protein comprising the second and third type 1 repeats of human TSP-1;
b) a chimeric protein comprising the multimerization domain of human COMP, the first type 2 repeat of human COMP, and the second and third type 1 repeats of human TSP-1;
c) a chimeric protein comprising the multimerization domain of human COMP, the first type 2 repeat of human COMP, and the second and third type 1 repeats of human TSP-1, but not the TGF-β activation region of human TSP-1;
d) a chimeric protein comprising the multimerization domain of human COMP, the procollagen region, and the first, second, and third type 1 repeats of human TSP-1; and
e) a chimeric protein comprising the three type 1 repeats of human TSP-2; and (6) a chimeric protein comprising the multimerization domain of human COMP, the first type 2 repeat of human COMP, and the three type 1 repeats of human TSP-2.

53. The method of claim 51 wherein the anti-angiogenic protein is administered locally at the site of one or more growths.


SEQUENCE LISTING (21 sequences)

SEQ ID NO: 1 - protein, 1152 aa, Homo sapiens
NRIPESGGDNSVFDIFELTGAARKGSGRRLVKGPDPSSPAFRIEDANLIPPVPDDKFQDLVDAVRTEKGFLLLASLRQMKKTRGTLLALERKDHSGQVFSVVSNGKAGTLDLSLTVQGKQHVVSVEEALLATGQWKSITLFVQEDRAQLYIDCEKMENAELDVPIQSVFTRDLASIARLRIAKGGVNDNFQGVLQNVRFVFGTTPEDILRNKGCSSSTSVLLTLDNNVVNGSSPAIRTNYIGHKTKDLQAICGISCDELSSMVLELRGLRTIVTTLQDSIRKVTEENKELANELRRPPLCYHNGVQYRNNEEWTVDSCTECHCQNSVTICKKVSCPIMPCSNATVPDGECCPRCWPSDSADDGWSPWSEWTSCSTSCGNGIQQRGRSCDSLNNRCEGSSVQTRTCHIQECDKRFKQDGGWSHWSPWSSCSVTCGDGVITRIRLCNSPSPQMNGKPCEGEARETKACKKDACPINGGWGPWSPWDICSVTCGGGVQKRSRLCNNPTPQFGGKDCVGDVTENQICNKQDCPIDGCLSNPCFAGVKCTSYPDGSWKCGACPPGYSGNGIQCTDVDECKEVPDACFNHNGEHRCENTDPGYNCLPCPPRFTGSQPFGQGVEHATANKQVCKPRNPCTDGTHDCNKNAKCNYLGHYSDPMYRCECKPGYAGNGIICGEDTDLDGWPNENLVCVANATYHCKKDNCPNLPNSGQEDYDKDGIGDACDDDDDNDKIPDDRDNCPFHYNPAQYDYDRDDVGDRCDNCPYNHNPDQADTDNNGEGDACAADIDGDGILNERDNCQYVYNVDQRDTDMDGVGDQCDNCPLEHNPDQLDSDSDRIGDTCDNNQDIDEDGHQNNLDNCPYVPNANQADHDKDGKGDACDHDDDNDGIPDDKDNCRLVPNPDQKDSDGDGRGDACKDDFDHDSVPDIDDICPENVDISETDFRRFQMIPLDPKGTSQNDPNWVVRHQGKELVQTVNCDPGLAVGYDEFNAVDFSGTFFINTERDDDYAGFVFGYQSSSRFYVVMWKQVTQSYWDTNPTRAQGYSGLSVKVVNSTTGPGEHLRNALWHTGNTPGQVRTLWHDPRHIGWKDFTAYRWRLSHRPKTGFIRVVMYEGKKIMADSGPIYDKTYAGGRLGLFVFSQEMVFFSDLKYECRDP

SEQ ID NO: 2 - protein, 1168 aa, Homo sapiens
MVWRLVLLALWVWPSTQAGHQDKDTTFDLFSISNINRKTIGAKQFRGPDPGVPAYRFVRFDYIPPVNADDLSKITKIMRQKEGFFLTAQLKQDGKSRGTLLALEGPGLSQRQFEIVSNGPADTLDLTYWIDGTRHVVSLEDVGLADSQWKNVTVQVAGETYSLHVGCDLIGPVALDEPFYEHLQAEKSRMYVAKGSARESHFRGLLQNVHLVFENSVEDILSKKGCQQGQGAEINAISENTETLRLGPHVTTEYVGPSSERRPEVCERSCEELGNMVQELSGLHVLVNQLSENLKRVSNDNQFLWELIGGPPKTRNMSACWQDGRFFAENETWVVDSCTTCTCKKFKTICHQITCPPATCDSFVEGECCPSCLHSVDGEEGWSPWAEWTQCSVTCGSGTQQRGRSCDVTSNTCLGPSIQTRACSLSKCDTRIRQDGGWSHWSPWSSCSVTCGVGNITRIRLCNSPVPQMGGKNCKGSGRETKACQGAPCPIDGRWSPWSPWSACTVTCAGGIRERTRVCNSPEPQYGGKACVGDVQERQMCNKRSCPVDGCLSNPCFPGAQCSSFPDGSWSCGFCPVGFLGNGTHCEDLDECALVPDICFSTSKVPRCVNTQPGFHCLPCPPRYRGNQPVGVGLEAAKTEKQVCEPENPCKDKTHNCHKHAECIYLGHFSDPMYKCECQTGYAGDGLICGEDSDLDGWPNLNLVCATNATYHCIKDNCPHLPNSGQEDFDKDGIGDACDDDDDNDGVTDEKDNCQLLFNPRQADYDKDEVGDRCDNCPYVHNPAQIDTDNNGEGDACSVDIDGDDVFNERDNCPYVYNTDQRDTDGDGVGDHCDNCPLVHNPDQTDVDNDLVGDQCDNNEDIDDDGHQNNQDNCPYISNANQADHDRDGQGDACDPDDDNDGVPDDRDNCRLVFNPDQEDLDGDGRGDICKDDFDNDNIPDIDDVCPENNAISETDFRNFQMVPLDPKGTTQIDPNWVIRHQGKELVQTANSDPGIAVGFDEFGSVDFSGTFYVNTDRDDDYAGFVFGYQSSSRFYVVMWKQVTQTYWEDQPTRAYGYSGVSLKVVNSTTGTGEHLRNALWHTGNTPGQVRTLWHDPRNIGWKDYTAYRWHLTPKTGYIRVLVHEGKQVMADSGPIYDQTYAGGRLGLFVFSQEMVYFSDLKYECRDI

SEQ ID NO: 3 - protein, 757 aa, Homo sapiens
MVPDTACVLLLTLAALGASGQGQSPLGSDLGPQMLRELQETNAALQDVRDWLRQQVREITFLKNTVMECDACGMQQSVRTGLPSVRPLLHCAPGFCFPGVACIQTESGGRCGPCPAGFTGNGSHCTDVNECNAHPCFPRVRCINTSPGFRCEACPPGYSGPTHQGVGLAFAKANKQVCTDINECETGQHNCVPNSVCINTRGSFQCGPCQPGFVGDQASGCQRGAQRFCPDGSPSECHEHADCVLERDGSRSCVCRVGWAGNGILCGRDTDLDGFPDEKLRCPEPQCRKDNCVTVPNSGQEDVDRDGIGDACDPDADGDGVPNEKDNCPLVRNPDQRNTDEDKWGDACDNCRSQKNDDQKDTDQDGRGDACDDDIDGDRIRNQADNCPRVPNSDQKDSDGDGIGDACDNCPQKSNPDQADVDHDFVGDACDSDQDQDGDGHQDSRDNCPTVPNSAQEDSDHDGQGDACDDDDDNDGVPDSRDNCRLVPNPGQEDADRDGVGDVCQDDFDADKVVDKIDVCPENAEVTLTDFRAFQTVVLDPEGDAQIDPNWVVLNQGREIVQTMNSDPGLAVGYTAFNGVDFEGTFHVNTVTDDDYAGFIFGYQDSSSFYVVMWKQMEQTYWQANPFRAVAEPGIQLKAVKSSTGPGEQLRNALWHTGDTESQVRLLWKDPRNVGWKDKKSYRWFLQHRPQVGYIRVRFYEGPELVADSNVVLDTTMRGGRLGVFCFSQENIIWANLRYRCNDTIPEDYETHQLRQA

SEQ ID NO: 4 - DNA, 755 bp, Artificial Sequence
  fusion gene
cagcacccagctccccgccaccgccatggtccccgacaccgcctgcgttcttctgctcaccctggctgccctcggcgcgtccggacagggccagagcccgttgggctcagacctgggcccgcagatgcttcgggaactgcaggaaaccaacgcggcgctgcaggacgtgcgggactggctgcggcagcaggtcagggagatcacgttcctgaaaaacacggtgatggagtgtgacgcgtgcgggatgcagcagtcagtacgcaccggcctacccagcgtgcggcccctgctccactgcgcgcccggcttctgcttccccggcgtggcctgcatccagacggagagcggcggccgctgcggcccctgccccgcgggcttcacgggcaacggctcgcactgcaccgacgtcgatggtggctggagccactggtccccgtggtcatcttgttctgtgacatgtggtgatggtgtgatcacaaggatccggctctgcaactctcccagcccccagatgaacgggaaaccctgtgaaggcgaagcgcgggagaccaaagcctgcaagaaagacgcctgccccatcaatggaggctggggtccttggtcaccatgggacatctgttctgtcacctgtggaggaggggtacagaaacgtagtcgtctctgcaacaaccccacaccccagtttggaggcaaggactgcgttggtgatgtaacagaaaaccagatctgcaacaagcaggactgtccaatctaga

SEQ ID NO: 5 - protein, 242 aa, Artificial Sequence
  chimeric protein
MVPDTACVLLLTLAALGASGQGQSPLGSDLGPQMLRELQETNAALQDVRDWLRQQVREITFLKNTVMECDACGMQQSVRTGLPSVRPLLHCAPGFCFPGVACIQTESGGRCGPCPAGFTGNGSHCTDVDGGWSHWSPWSSCSVTCGDGVITRIRLCNSPSPQMNGKPCEGEARETKACKKDACPINGGWGPWSPWDICSVTCGGGVQKRSRLCNNPTPQFGGKDCVGDVTENQICNKQDCPI

SEQ ID NO: 6 - DNA, 925 bp, Artificial Sequence
  fusion gene
cagcacccagctccccgccaccgccatggtccccgacaccgcctgcgttcttctgctcaccctggctgccctcggcgcgtccggacagggccagagcccgttgggctcagacctgggcccgcagatgcttcgggaactgcaggaaaccaacgcggcgctgcaggacgtgcgggactggctgcggcagcaggtcagggagatcacgttcctgaaaaacacggtgatggagtgtgacgcgtgcgggatgcagcagtcagtacgcaccggcctacccagcgtgcggcccctgctccactgcgcgcccggcttctgcttccccggcgtggcctgcatccagacggagagcggcggccgctgcggcccctgccccgcgggcttcacgggcaacggctcgcactgcaccgacgtcgaggagggctggtctccgtgggcagagtggacccagtgctccgtgacgtgtggctctgggacccagcagagaggccggtcctgtgacgtcaccagcaacacctgcttggggccctcgatccagacacgggcttgcagtctgagcaagtgtgacacccgcatccggcaggacggcggctggagccactggtcaccttggtcttcatgctctgtgacctgtggagttggcaatatcacacgcatccgtctctgcaactccccagtgccccagatggggggcaagaattgcaaagggagtggccgggagaccaaagcctgccagggcgccccatgcccaatcgatggccgctggagcccctggtccccgtggtcggcctgcactgtcacctgtgccggtgggatccgggagcgcacccgggtctgcaacagccctgagcctcagtacggagggaaggcctgcgtgggggatgtgcaggagcgtcagatgtgcaacaagaggagctgccccgtgtctaga

SEQ ID NO: 7 - protein, 300 aa, Artificial Sequence
  chimeric protein
MVPDTACVLLLTLAALGASGQGQSPLGSDLGPQMLRELQETNAALQDVRDWLRQQVREITFLKNTVMECDACGMQQSVRTGLPSVRPLLHCAPGFCFPGVACIQTESGGRCGPCPAGFTGNGSHCTDVEEGWSPWAEWTQCSVTCGSGTQQRGRSCDVTSNTCLGPSIQTRACSLSKCDTRIRQDGGWSHWSPWSSCSVTCGVGNITRIRLCNSPVPQMGGKNCKGSGRETKACQGAPCPIDGRWSPWSPWSACTVTCAGGIRERTRVCNSPEPQYGGKACVGDVQERQMCNKRSCPVSR

SEQ ID NO: 8 - protein, 7 aa, Homo sapiens
NGVQYRN

SEQ ID NO: 9 - protein, 6 aa, Homo sapiens
CSVTCG

SEQ ID NO: 10 - protein, 7 aa, Homo sapiens
  VARIANT 3, 6: Xaa = Any Amino Acid
WSXWSXW

SEQ ID NO: 11 - protein, 6 aa, Homo sapiens
GGWSHW

SEQ ID NO: 12 - protein, 19 aa, Homo sapiens
SPWDICSVTCGGGVQKRSR

SEQ ID NO: 13 - protein, 33 aa, Homo sapiens
  VARIANT 6, 7, 8, 9, 10, 11, 13, 14, 15, 16, 17, 18, 19, 20: Xaa = Any Amino Acid
DVDECXXXXXXCXXXXXXXXCENTDPGYNCLPC

SEQ ID NO: 14 - DNA, 27 bp, Artificial Sequence
  oligonucleotide
gatgacgtcactgaagagaacaaagag

SEQ ID NO: 15 - DNA, 22 bp, Artificial Sequence
  oligonucleotide
gatgacgtccacagccaccgcg

SEQ ID NO: 16 - DNA, 28 bp, Artificial Sequence
  oligonucleotide
gattctagactacttggaggcagtcatg

SEQ ID NO: 17 - DNA, 27 bp, Artificial Sequence
  oligonucleotide
gatgacgtcgatggtggctggagccac

SEQ ID NO: 18 - DNA, 25 bp, Artificial Sequence
  oligonucleotide
gatctagattggacagtcctgcttg

SEQ ID NO: 19 - DNA, 27 bp, Artificial Sequence
  oligonucleotide
gatgacgtcgaggagggctggtctccg

SEQ ID NO: 20 - DNA, 27 bp, Artificial Sequence
  oligonucleotide
gatctagacacggggcagctcctcttg

SEQ ID NO: 21 - protein, 15 aa, Homo sapiens
SDLGPQMLRELQETN